(19) United States
(12) Patent Application Publication
(10) Pub. No.: US 2023/0218736 A1
(43) Pub. Date: Jul. 13, 2023

(54) Cocktail Vaccine of Recombinant African Swine Fever Virus (ASFV) Antigen and Use Thereof

(71) Applicant: Lanzhou Veterinary Research Institute, Chinese Academy of Agricultural Sciences, Lanzhou (CN)

(72) Inventors: Junjun SHAO, Lanzhou (CN); Guanglei ZHANG, Lanzhou (CN); Wei LIU, Lanzhou (CN); Xiaxia LIANG, Lanzhou (CN); Huiyun CHANG, Lanzhou (CN)

(73) Assignee: Lanzhou Veterinary Research Institute, Chinese Academy of Agricultural Sciences, Lanzhou, GS (CN)

(21) Appl. No.: 17/948,752

(22) Filed: Sep. 20, 2022

(30) Foreign Application Priority Data
Jan. 11, 2022 (CN) 202210024618 .6

(51) Int. Cl. A61K 39/12; C07K 14/005; A61K 39/295; A61P 31/20

(52) U.S. Cl. CPC A61K 39/12; C07K 14/005; A61K 39/295; A61P 31/20; C12N 2710/12034; C12N 2710/12022; A61K 2039/552

(57) ABSTRACT

The present disclosure relates to a cocktail vaccine of a recombinant African swine fever virus (ASFV) antigen and use thereof, and belongs to the field of biotechnology pharmacy. The cocktail vaccine is prepared by combination of recombinant proteins p30-modified p54, p72 epitope-N-segment amino acid sequence of pE248R, and N-terminal amino acid sequence of CD2v-C-terminal amino acid sequence of pEP153R with an adjuvant. The present disclosure provides a method for expressing and purifying the three recombinant proteins, as well as a ratio of each recombinant protein and the adjuvant for preparing the cocktail vaccine.

### CROSS REFERENCE TO RELATED APPLICATION

This patent application claims the benefit and priority of Chinese Patent Application No. 202210024618.6, entitled “COCKTAIL VACCINE OF RECOMBINANT AFRICAN SWINE FEVER VIRUS (ASFV) ANTIGEN AND USE THEREOF” filed on Jan. 11, 2022, the disclosure of which is incorporated by reference herein in its entirety as part of the present application.

### REFERENCE TO SEQUENCE LISTING

A computer readable XML file entitled “HLP20220602625_20221013”, that was created on Oct. 13, 2022, with a file size of about 13,027 bytes, contains the sequence listing for this application, has been filed with this application, and is hereby incorporated by reference in its entirety.

### TECHNICAL FIELD

The present disclosure belongs to the field of biotechnology pharmacy, and in particular relates to a cocktail vaccine of a recombinant African swine fever virus (ASFV) antigen and use thereof.

### BACKGROUND ART

African swine fever (ASF) is an acute and severe infectious disease caused by African swine fever virus (ASFV) in pigs, with a mortality rate up to 100%. Currently, there is no commercial available vaccine for the ASF. The World Organization for Animal Health (OIE) lists ASF as an animal disease that must be notified, and China lists ASF as the Class I animal infectious disease that must be prevented and controlled emphatically. Since the discovery of ASFV, researches on vaccines have never stopped. However, due to the large genome, numerous encoded proteins and complex structure of ASFV, the mechanism of immunity and infection on ASFV is unclear, and so far there is no safe and effective vaccine available for the ASFV. Studies on ASF vaccines have shown that: (1) inactivated ASF vaccines cannot provide immune protection; (2) live attenuated ASF vaccines can prevent infection against homologous or heterologous strains, but can cause adverse reactions and has a potential risk of virulence recovery; (3) ASF subunit vaccines can induce production of neutralizing antibodies, but cannot offer complete protection to pigs against challenge with the virulent virus. The ASF recombinant subunit vaccines have already become a promising field on development of ASF vaccine, due to the recombinant subunit vaccines are very safety. It is in need of designing a new multi-component subunit vaccine involving multiple protective antigens to improve the immune protection level of the subunit vaccine.

The ASFV genome has a size of 170 kb to 194 kb, includes 150 to 167 open reading frames (ORFs), and encodes more than 50 structural proteins and more than 100 non-structural proteins. Studies have confirmed that viral proteins including p30, p54, p72, CD2v and pEP153R can induce neutralizing antibodies, where the anti-p54 and -p30 antibodies can inhibit the adsorption and internalization of viruses, the anti-p72 antibodies can also inhibit the adsorption of ASFV to porcine alveolar macrophage (PAM) cells. In addition, recombinant protein CD2v can induce immune protection against virus infection. pEP153R as a multifunctional protein induces specific antibodies, which can provide partial immune protection against homologous virus infection. In conclusion, in view of the complex immune protection mechanism of ASFV, it is particularly important to design a subunit vaccine consist of multi-component antigens, which not only broaden the antigen spectrum, but enhance the level of neutralizing antibodies. This is one of the important strategies to solve the undesirable immune protection effect of the ASF subunit vaccine.

### SUMMARY

To develop a safe and effective ASF vaccine, in the present disclosure, three recombinant ASFV antigen fusion proteins are obtained by gene fusion and tandem expression, namely p30-modified p54, p72 epitope-N terminal domain of pE248R, and N terminal domain of CD2v-C terminal domain of pEP153R, respectively. The three recombinant proteins are emulsified with an adjuvant in a certain ratio to formulate a cocktail vaccine, and the cocktail vaccine can induce anti-ASFV neutralizing antibodies and cellular immune response after immunizing pigs, suggesting that this vaccine is a promising ASF subunit vaccine for development and use.

The present disclosure adopts the following specific technical protocol.

The present disclosure provides a cocktail vaccine based on the recombinant ASFV antigens, where the cocktail vaccine includes two or more of three recombinant ASFV antigen fusion proteins, including PM, PPE, and CPE as active ingredients;

the PM is formed by fusion of ASFV p30 and modified p54 through a Linker, with a general formula of p30-(Linker)₃-mp54;

the PPE is formed by fusion of four verified epitopes of ASFV p72 and the N-terminal amino acid sequence of pE248R through the Linker, with a general formula of p72 epitope 1-(Linker)₃-p72 epitope 2-(Linker)₃-p72 epitope 3-(Linker)₃-p72 epitope 4-(Linker)₃-N-terminal amino acid sequence of pE248R;

the CPE is formed by fusion of the N-terminal amino acid sequence of CD2v and the C-terminal amino acid sequence of pEP153R through the Linker, with a general formula of N-terminal amino acid sequence of CD2v-(Linker)₂-C-terminal amino acid sequence of pEP153R; and

the Linker has a sequence of GGGGS (SEQ ID NO:7).

In some embodiments, the PM may have the amino acid sequence set forth in SEQ ID NO: 4; the PPE may have the amino acid sequence set forth in SEQ ID NO: 5; and the CPE may have the amino acid sequence set forth in SEQ ID NO: 6.

In some embodiments, the nucleotide sequence encoding the PM is set forth in SEQ ID NO: 1; the nucleotide sequence encoding the PPE is set forth in SEQ ID NO: 2; and the nucleotide sequence encoding the CPE is set forth in SEQ ID NO: 3.

In some embodiments, the cocktail vaccine may include the three recombinant ASFV antigen fusion proteins PM, PPE, and CPE as the active ingredients. In some other embodiments, the cocktail vaccine may further include an adjuvant, which is prepared by mixing the PM, the PPE, and the CPE with the adjuvant. In some embodiments, the three recombinant ASFV antigen fusion proteins PM, PPE, and CPE may be mixed in a mass ratio of 1:1:2 to obtain an antigen mixture; and the antigen mixture may be emulsified into the cocktail vaccine with equal quality MONTANIDE™ISA 206 VG adjuvant.

The present disclosure further provides use of the cocktail vaccine in preparation of a drug for treating/preventing ASFV infection.

By adopting the foregoing technical solutions, the present disclosure achieves the following advantages:

1. The three recombinant ASFV antigen fusion proteins of the present disclosure each can be induced and expressed in a prokaryotic expression system (E. coli), with high expression levels and easy purification.

2. The cocktail vaccine made of the three recombinant ASFV antigen fusion proteins includes a total of six ASFV proteins, with a more comprehensive antigen spectrum.

3. The ASF cocktail vaccine can induce high levels of specific antibodies and cellular immunity in vaccinated pigs. Meanwhile, it is confirmed by the virus neutralization test that the antibodies induced by the cocktail vaccine have a significant inhibitory effect on ASFV infection of host cells in vitro, suggesting a desirable immune protection effect.

### BRIEF DESCRIPTION OF THE DRAWINGS

FIG. 1 shows an SDS-PAGE result after purification of three recombinant ASFV antigens in examples of the present disclosure; where Lane 1: Protein Molecular Weight Standard (Marker); Lane 2: Purified PM; Lane 3: Purified PPE; Lane 4: Purified CPE;

FIGS. 2A and 2B show identification results of Western blotting of the three recombinant proteins using a mouse anti-6×His monoclonal antibody (2A) and an anti-ASFV positive serum (2B) in examples of the present disclosure; where Lane 1: Purified PM; Lane 2: Purified PPE; Lane 3: Purified CPE;

FIGS. 3A, 3B, 3C, 3D,3D, 3E, and 3F show fluctuation dynamics in specific IgG of p30 (3A), p54 (3B), p72 (3C), pE248R (3D), CD2v (3E), and pEp153R (3F) in serum at different time points from pigs after primary immunization in examples of the present disclosure;

FIGS. 4A and 4B show a proliferation percentage of peripheral blood mononuclear lymphocytes (PBMCs) isolated from the pigs on 35 d after the primary immunization, which stimulated with the inactivated ASFV in examples of the present disclosure;

FIGS. 5A, 5B, and 5C show a percentage of CD8+T cells secreting IFN-γ(4A), IL-2(4B), and TNF-α(4C) in the total number of T lymphocytes from the pigs on 35 d after the primary immunization, which are stimulated with the inactivated ASFV in examples of the present disclosure;

FIG. 6 shows indirect immunofluorescence results of ASFV in porcine alveolar macrophages (PAM) inoculated with the incubation mixture of the immunized pig serum and ASFV that were detected with mouse anti-p30 monoclonal antibody in examples of the present disclosure; and

FIGS. 7A and 7B show copy numbers of ASFV genome (7A) and a virus neutralization rate (7B) in PAM infected with the mixture of ASFV and the serum from pigs vaccinated with the ASF cocktail vaccine in examples of the present disclosure.

### DETAILED DESCRIPTION OF THE EMBODIMENTS

Inventors of the present disclosure found that three recombinant ASFV antigen fusion proteins are obtained by gene fusion and tandem expression, namely p30-modified p54, p72 epitope-N-terminal amino acid sequence of pE248R, and N-terminal amino acid sequence of CD2v-C-terminal amino acid sequence of pEP153R. The three recombinant proteins are combined with an adjuvant in a certain ratio to formulate a cocktail vaccine, and the cocktail vaccine can induce neutralizing antibodies and cellular immune response in pigs after immunizing, suggesting that this vaccine type is a promising ASF subunit vaccine for use. The three recombinant ASFV antigen fusion proteins are as follows:

(1) a recombinant ASFV antigen fusion protein PM, the recombinant protein is formed by fusion of ASFV p30 and modified p54 (mp54) through a linker peptide (Linker), with a general formula of p30-(Linker)₃-mp54;

(2) a recombinant ASFV antigen fusion protein PPE, the recombinant protein is formed by fusion of four epitopes of ASFV p72 and an N-terminal amino acid sequence of pE248R through the linker peptide (Linker), with a general formula of p72 epitope 1-(Linker)₃-p72 epitope 2-(Linker)₃-p72 epitope 3-(Linker)₃-p72 epitope 4-(Linker)₃-N-terminal amino acid sequence of pE248R;

(3) a recombinant ASFV antigen fusion protein CPE, the recombinant protein is formed by fusion of an N-terminal amino acid sequence of CD2v and a C-terminal amino acid sequence of pEP153R through the linker peptide (Linker), with a general formula of N-terminal amino acid sequence of CD2v-(Linker)₂-C-terminal amino acid sequence of pEP153R; and the Linker has a sequence of GGGGS.

In the present embodiments, the recombinant ASFV antigen fusion proteins PM, PPE, and CPE have amino acid sequences set forth in SEQ ID NO: 4, SEQ ID NO: 5, and SEQ ID NO: 6, respectively.

In the present embodiments, nucleotide sequences encoding the recombinant ASFV antigen fusion proteins PM, PPE, and CPE are set forth in SEQ ID NO: 1, SEQ ID NO: 2, and SEQ ID NO: 3, respectively.

A method for expression, purification, and identification of the recombinant proteins includes:

(1) vector construction: constructing a recombinant expression vector, where the expression vector is formed by linking the nucleotide sequence encoding the recombinant protein in a modifiable manner by a backbone plasmid; in some embodiments, the backbone plasmid is pET-30a (+);

(2) transformation and screening of positive clones: transforming the recombinant expression vector in step (1) into competent cells of host bacteria, and obtaining recombinant bacteria capable of expressing a target protein after induction and SDS-PAGE identification;

(3) induced expression: transferring positive recombinant bacteria obtained in step (2) to grow to a certain concentration, and adding IPTG to induce expression of the recombinant protein;

(4) protein purification: collecting bacterial cells in step (3), and conducting ultrasonication, dissolution of inclusion bodies, purification by Ni-NTA affinity chromatography, and dialysis renaturation to obtain a target protein; and

(5) identification of the recombinant protein: identifying the target protein obtained in step (4) by SDS-PAGE and Western blotting.

The present disclosure further provides a method for preparing a cocktail vaccine of recombinant ASFV antigens with the recombinant proteins, including the following steps:

determination of a protein concentration: determining a concentration of a purified recombinant protein by a Bradford method;

preparation of an antigen mixture: diluting recombinant proteins to the same concentration and mixing in a certain ratio; and

emulsification of a vaccine: conducting emulsification on an antigen mixture obtained in step (2) with a MONTANIDE™ ISA 206 VG adjuvant (50 g: 50 g) to prepare a vaccine.

To better understand the present disclosure, the content of the present disclosure is further described below in details in conjunction with the examples, but the claimed content of the present disclosure is not limited to the following examples.

I. Experimental Materials

1.1. Cells and Viruses

BL21(DE3) competent cells were purchased from Sangon (Shanghai); porcine alveolar macrophages were isolated from lungs of healthy pig and stored in liquid nitrogen by the research group; and ASFV (CN/SC/2019) strains were isolated and preserved by the Lanzhou Veterinary Research Institute of the Chinese Academy of Agricultural Sciences (National ASF Regional Laboratory).

1.2. Experimental Animals

6-week-old healthy female piglets were purchased from a farm in Kangle County, Gansu Province.

II. Experimental methods and results

2.1. Construction of a Recombinant Plasmid

(1) According to an existing ASFV, ASFV-SY18 (accession number: MH766894.1) in GenBank, the amino acid sequences of p30 and p54 were selected, where a transmembrane region His30-Phe52 of p54 replaced a linking peptide (GGGGS)₃ to obtain mP54, and the mP54 was ligated with a C-terminal amino acid sequence of p30 through the linking (GGGGS)₃, to obtain an amino acid sequence of a recombinant fusion protein PM. (2) According to the 4 epitopes located in ASFV p72 (epitope IDs: 141844, 141941, 141989, and 142069) that have been verified in the Immune Epitope Database (IEDB) database, the epitopes were ligated in series through the (GGGGS)₃ flexible Linker, and the ligated p72 epitope was ligated with an N-terminal amino acid sequence (Metl-Lysl98) of pE248R (protein ID: AYW34102.1) of ASFV, ASFV-SY18, through the (GGGGS)₃ flexible Linker in series, to obtain an amino acid sequence of a recombinant fusion protein PPE. (3) The N-terminal amino acid sequence (Asp17-Tyr206) of ASFV-SY18 CD2v (Protein ID: AYW34030.1) and the C-terminal amino acid sequence (Asn49-Lys158) of pEP153R (Protein ID: AYW34029.1) were ligated in series by a (GGGGS)₂ flexible Linker, to obtain an amino acid sequence of a recombinant fusion protein CPE. The amino acid sequences of the above three recombinant fusion proteins were converted into corresponding nucleotide sequences according to the codon preference of E. coli, and specific enzyme cleavage sites BamH I and Xho I were introduced at 5′- and 3′- ends of each nucleotide sequence; the obtained nucleotide sequences were entrusted to Nanjing GenScript Biotechnology Co., Ltd. to synthesize and clone into a pET-30a(+) expression vector, to obtain recombinant expression plasmids pET-30a(+)-PM, pET-30a(+)-PPE, and pET-30a(+)-CPE. The results of gene sequencing showed that the sequences between BamH I and Xho I on the three recombinant plasmids had 100% homology with the designed target genes, and the reading frame was completely correct.

2.2. Expression and Purification of Recombinant Proteins

The three recombinant expression plasmids were transformed into E. coli BL21 (DE3) competent cells separately, and positive clones were selected and inoculated into 5 mL of LB (kan⁺), incubated at 37° C. for 8 h at 220 rpm, and then inoculated into 1 L of LB (kan⁺) at 1:100, cultured at 37° C. at 220 rpm, when the OD₆₀₀ of the bacterial solution reached 0.4 to 0.6, a final concentration of 1 mM of IPTG was added, and continued to culture for 4 h at 37° C. at 220 rpm. Bacteria were collected by centrifugation at 7,000 rpm for 6 min, resuspended in 50 mL of a binding buffer (300 mM NaCl, 20 mM NaH₂PO₄, and 5 mM imidazole; at pH 8.0), sonicated for 40 min (in ice bath), and centrifuged at 10,000 rpm for 25 min to collect precipitation (inclusion bodies). The inclusion bodies were dissolved with a binding buffer containing 8 M urea, centrifuged at 10,000 rpm for 25 min, a supernatant was collected, transferred to a Ni-NTA affinity chromatography column, and bound at 4° C. for 1.5 h. The impure proteins were washed with 10 column volumes of washing buffer (8 M urea, 300 mM NaCl, 20 mM NaH₂PO₄, and 20 mM imidazole; at pH 8.0), and a target recombinant protein was eluted with an appropriate amount of elution buffer (8 M urea, 300 mM NaCl, 20 mM NaH₂PO₄, and 500 mM imidazole; at pH 8.0). The obtained recombinant protein was dialyzed and renatured by 8 M, 6 M, 4 M, 2 M and 0 M of dialysate (20 mM NaH₂PO₄, 300 mM NaCl, 2 mM β-mercaptoethanol, 0.4% arginine, and 10% glycerol; at pH 7.5) in sequence, each gradient was dialyzed for 8 h, and a small amount of renatured recombinant protein was taken for SDS-PAGE. The results showed that the renatured recombinant proteins PM, PPE, and CPE were 56 kDa, 46 kDa, and 32 kDa, respectively, as shown in FIG. 1.

2.3. Western Blotting Identification of Recombinant Protein

80 μL of the three recombinant proteins were mixed with a 5× loading buffer and boiled for 10 min separately; after loading, SDS-PAGE was conducted (80 V for 30 min, 120 V for 1 h and 10 min), and then electrotransferred to a polyvinylidene fluoride (PVDF) membrane, and blocked in a 1× PBST solution with 5% nonfat dry milk for 2 h at room temperature. Primary antibody incubation: the blocked PVDF membrane was incubated with a mouse anti-6×His monoclonal antibody (with 1:5000-fold dilution) and anti-ASFV positive serum (with 1:300-fold dilution) overnight at 4° C., and washed with 1× PBST for 6 times. Secondary antibody incubation: peroxidase-labeled goat anti-mouse IgG (with 1:5000-fold dilution) and peroxidase-labeled goat anti-pig IgG (with 1:5000-fold dilution) were added to the corresponding PVDF membranes, incubated at room temperature for 1 h, and washed with 1× PBST for 6 times. Color development: an ECL luminescent solution was prepared and spread evenly on the PVDF membranes; and image acquisition was conducted with a multi-function imager.

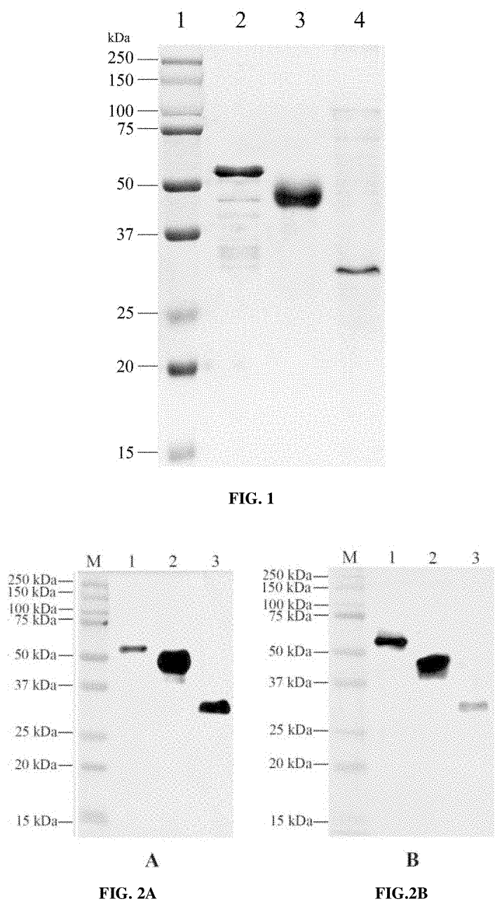

Results and analysis: the results of recombinant protein identification by Western blotting were shown in FIGS. 2A and 2B; the three recombinant proteins could be recognized by the ANTI-6×His monoclonal antibody and had a desirable reactivity with the ASFV positive serum, which had a potential value for use.

2.4. Vaccine Preparation and Immunization Scheme

The concentration of three recombinant proteins was separately determined by a Bradford method: 600 μg of the PM, 600 μg of the PPE, and 1,200 μg of the CPE were mixed and diluted to a total volume of 4 mL, and then mixed with a MONTANIDE™ ISA 206 VG adjuvant (50 g: 50 g) for emulsification to prepare a water/oil/water vaccine (W/O/W). Seven 6-week-old female piglets were randomly divided into 2 groups (4 in an experimental group and 3 in a control group); the prepared vaccine was injected intramuscularly, according to immunization groups and immunization dosages in the following table, with primary immunization for 1 time and booster immunization for 1 time after 21 d.

| | Immune | Injection |
|---|---|---|
| Group | component/head | volume mL/head |
| Experimental | 150 μg PM + 150 μg | 2 |
| group 1 | PPE + 300 μg CPE | |
| Control group | PBS | 2 |

Results and analysis: a standard curve was established according to the OD value of the different concentrations of protein standards at 592 nm. A linear equation, y=0.00049964x+0.28323, R²=0.993, was obtained according to the standard curve. The concentrations of the of the recombinant proteins PM, PPE and CPE were obtained according to the linear equation that were 677.1 μg/mL, 896.2 μg/mL, and 494.1 μg/mL, respectively.

2.5. Evaluation of Immune Effect

2.5.1. Antigen-Specific IgG Detection

Blood was collected from all experimental pigs before and 14, 21, 35 and 42 d after immunization, and the serum was separated by centrifugation at 4,000 rpm for 10 min. Detection of specific antibodies of immune sera was conducted by laboratory established indirect ELISA based on the recombinant proteins including p30, p54, p72, pE248R, CD2v, and pEP153R, respectively.

Detection procedure: the 96-well microtiter plate were separately coating with the purified recombinant p30 (0.125 μg/mL), p54 (0.5 μg/mL), p72 (1 μg/mL), pE248R (1 μg/mL), CD2v (2 μg/mL), and pEP153R (2 μg/mL) to overnight at 4° C., 100 μL/well; coating solution was discarded, blocking solution (5% nonfat dry milk, 200 μL/well) was added and incubated at 37° C. for 2 h; the blocking solution was discarded, serum samples diluted at 1:100 were added, 100 L/well, and then incubated at 37° C. for 1 h; the liquid was discarded and the plate was washed 5 times with 1×PBST; 1:10000-diluted horseradish peroxidase (HRP)-labeled goat anti-pig IgG was added, 100 μL/well, and then incubated at 37° C. for 1 h; the liquid was discarded and the plate was washed 5 times with 1×PBST; then TMB substrate was added, 100 μL/well, and reacted at room temperature for 10 min in the dark; stop solution (2 M H₂SO₄) was added, 100 VL/well, and the OD value was measured at 450 nm.

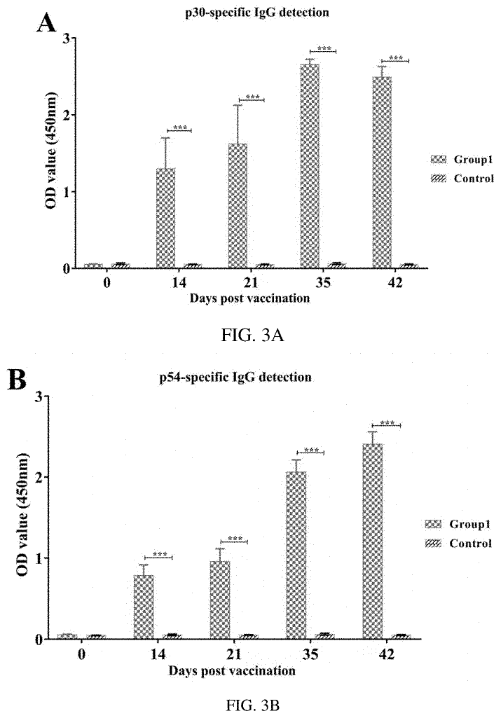

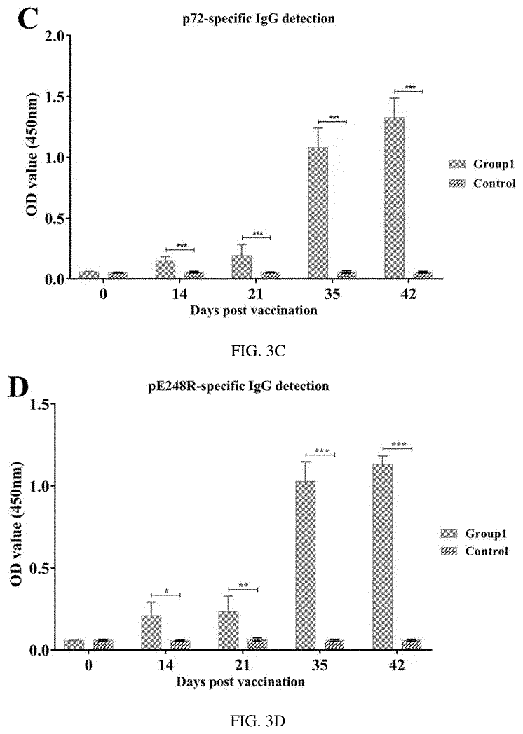

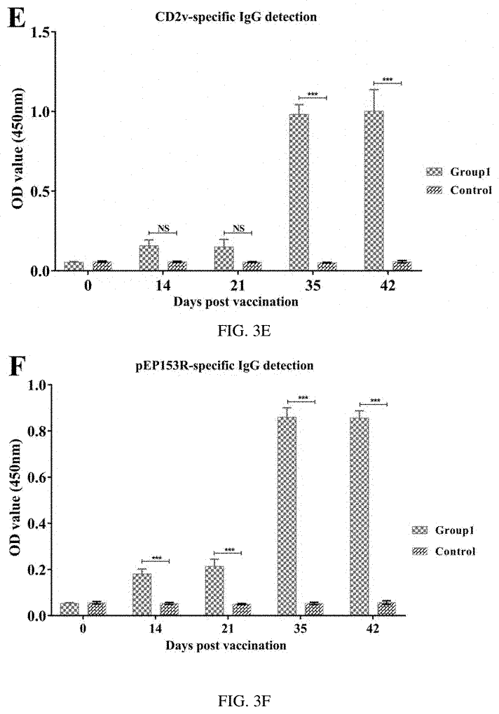

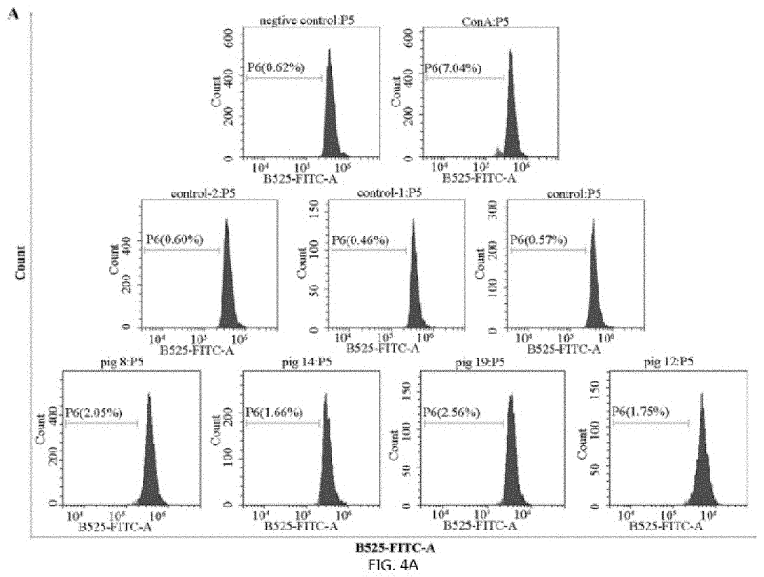

As shown in FIGS. 3A, 3B, 3C, 3D, 3E, and 3F, compared with the PBS group, the specific IgG antibodies against p30, p54, p72, pE248R, CD2v and pEP153R were initiated in the cocktail vaccine group on 7 d after immunization, and then the antibody titers increased significantly after the booster immunization. Among all the tested antibodies, specific IgG antibodies against p30 and p54 had the highest levels, followed by specific IgG antibodies against p72 and pE248R, and specific IgG antibodies against CD2v and pEP153R had the lowest levels, but all these antibodies were significantly higher than the PBS control (p<0.001). The results showed that the cocktail vaccine had desirable immunogenicity and could induce a higher level of specific IgG in pigs after immunizing.

2.5.2. Lymphocyte Proliferation Assay

42 d after Immunization, 5 mL of Anticoagulant Blood was Collected from Each Immunized Pig and diluted with an equal volume of sterile PBS, and then the anticoagulant blood was slowly and lightly added to a 50 mL centrifuge tube containing 10 mL of lymphocyte separation medium (Ficoll-paque, 1.077 g/ml); centrifugation was conducted at 800×g for 30 min (at room temperature) in a horizontal rotor centrifuge; the buffy coat (peripheral blood mononuclear lymphocyte cells, PBMCs) was transferred to a new centrifuge tube, cells were resuspended in 20 mL of sterile PBS, centrifuged at 250×g for 10 min, and the supernatant was discarded, the cell pellet was repeated once according to mention-above; the cells were resuspended in 0.5 mL PBS, and mixed evenly and quickly with 0.5 mL of 2.5 μM of CFSE working solution; the cell suspension was placed in a water bath at 37° C. for 10 min, and shaken once every 2 min; added with 5 mL of RPMI-1640, centrifuged at 1,500 rpm for 5 min to discard the supernatant, and the step was repeated once; the PBMCs was resuspended in RPMI-1640 containing 10% FBS, adjusted to 1×10⁶ cells/mL, and plated into a 24-well plate, 1 mL/well. The inactivated ASFV (virus titer is 10⁵ HAD₅₀) was added to each well of the experimental group, and a normal cell group, a non-stimulatory substance group and ConA (5 μg/mL) stimulation group were set up, as blank control, negative control and positive control, respectively, and all group were cultured at 37° C., 5% CO₂ for 72 h; the cells were collected by centrifugation at 1,500 rpm for 5 min, washed twice with PBS and resuspended in 100 μL PBS; fluorescence intensity in the PBMCs was measured by flow cytometry. The cell populations with lower fluorescence intensity were circled based on the negative control, and their percentages in total lymphocytes were calculated.

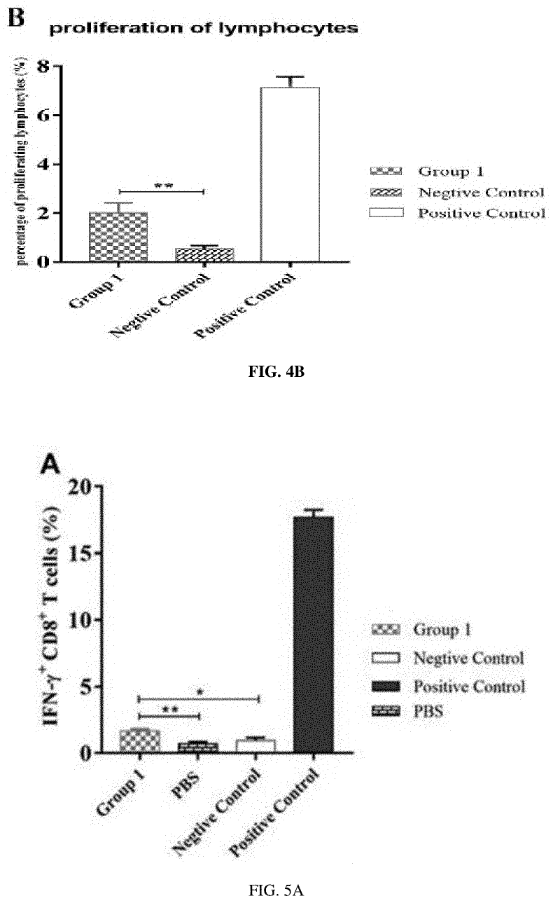

As shown in FIGS. 4A and 4B, after stimulation with the inactivated ASFV, the proportions of cells with low fluorescence intensity in the PBMCs isolated from the cocktail vaccine immunization group were significantly higher than that in the blank control and the PBS groups (p<0.01), namely the lymphocytes in the experimental group proliferated significantly after stimulation. The results showed that the cocktail vaccine could significantly activate cellular immunity and induce desirable immune memory.

2.5.3. Detection of Antigen-Specific CD8+T Lymphocytes

PBMCs were separated as described in 2.6, adjusted to a cell density of 1×10⁶ cells/mL, and inoculated in a 24-well plate, 1 mL per well. Cells from each well of the experimental group was added with inactivated ASFV (virus tier was 10⁵ HAD₅₀), where wells without stimulant were used as negative control, and wells added with 25 μg/mL phorbol ester and 1 μg/mL ionomycin were used as positive control, and cultured at 37° C., 5% CO₂ for 40 h, and then added with monensin, 1.7 μg/mL/well, and continued to incubate at 37° C., 5% CO₂ for 8 h. All cells in each well were collected, washed twice with PBS, resuspended in 100 μL of PBS. And then added with PerCP-Cy5.5-labeled anti-CD3 monoclonal antibody and PE-labeled anti-CD8 monoclonal antibody (1μ1 each antibody), and stained at 4° C. for 30 min. The cells was washed twice with PBS by centrifugation at 1,500 rpm for 5 min, and then resuspended with 500 μL of a fixative according to instructions of the Fixaion and Permeabiliation solution and kept for 20 min at room temperature in the dark. The cells were washed once with 1 mL of 1×Perm/Wash, and the cells were collected by centrifugation at 1,500 rpm for 5 min. The cell pellets were resuspended with 1.5 mL of the 1×Perm/Wash and incubated for 5 min at room temperature in the dark. The cells were collected by centrifugation at 1,500 rpm for 5 min, and resuspended in 100 μL of the 1×Perm/Wash. The cells were stained with AF700-labeled anti-IL-2, Pecy7-labeled anti-TNF-α and AF647-labeled anti-IFN-γ monoclonal antibodies at 4° C. in the dark for 30 min. The cells were washed twice with the 1×Perm/Wash by centrifugation, and resuspended in 100 μL of PBS containing 2% FBS for flow cytometry analysis to determine the percentages of the CD8+T cells positive for IFN-γ, IL-2 and TNF-α in the T lymphocytes.

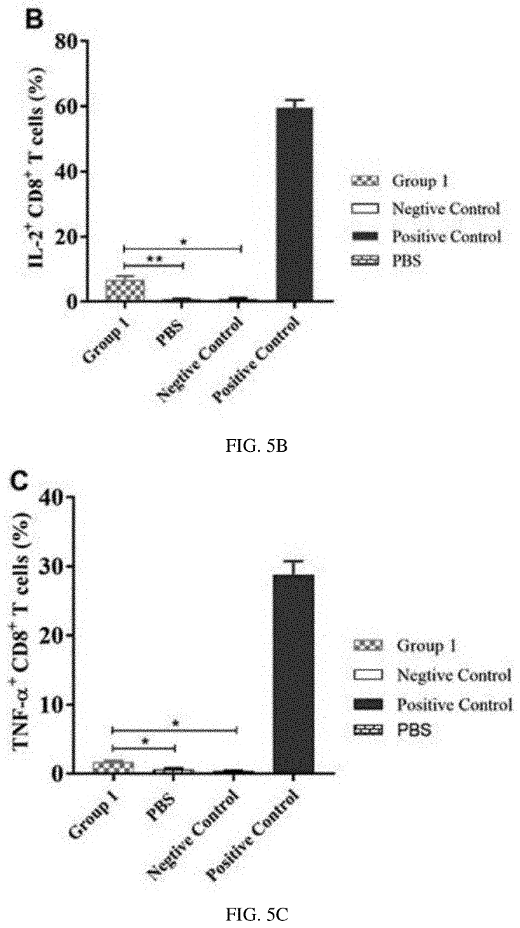

As shown in FIGS. 5A, 5B, and 5C, the proportion of CD8+T cells secreting IFN-γ, IL-2 and TNF-α in the PBMCs from the cocktail vaccine immunization group was significantly higher than that of the negative control and PBS groups after stimulation with inactivated ASFV (p<0.01), with the highest proportion in IL-2+CD8+T cells. The results proved that the cocktail vaccine induced the cellular immune response by activation of T lymphocytes after immunization, and to generate memory T lymphocytes.

2.5.4. Indirect Immunofluorescence Assay

On 42 d after immunization, blood was collected from each immunized pig to prepare serum; the immune serum was diluted at 1:5 and then inactivated at 56° C. for 30 min. The serum was mixed with an equal volume of ASFV (CN/SC/2019) (MOI=0.01), and incubated at 37° C. overnight. The monolayer of PAM cells was inoculated with the mixture of the immune serum and virus (24-well plate), 200 μL per well, at 37° C., 5% CO₂ for 1 h, and shaken gently every 10 min, The mixture of serum and virus was discarded and washed 3 times with PBS. Each well was added with 0.5 mL of RPMI-1640 containing 5% FBS and incubated for 48 h at 37° C., 5% CO₂. The cells were washed 3 times with PBS, added with 4% paraformaldehyde (0.5 mL/well), and incubated at 4° C. for 1 h; the paraformaldehyde was discarded and each well was added with 0.5 mL of 0.25% triton X-100 and allowed to stand for 10 min at room temperature; the cells were washed with PBS 3 times, 3 min in each time, and mixed on a micro shaker; 1 mL of 5% BSA was added to each well to block for 60 min; the blocking solution was discarded, each well was added with 0.5 mL of a mouse anti-p30 monoclonal antibody diluted at 1:2000, and incubated at 37° C. for 60 min; the liquid in the well was discarded and washed 3 times with PBS, each well was added with 0.5 mL of 1:500-diluted TRITC-labeled goat anti-mouse IgG in the dark, and incubated at 37° C. for 60 min; the liquid in the wells was discarded and washed 3 times with PBS, each well was added with two drops of DAPI and supplemented with 0.5 mL PBS, allowed to stand for 5 min at room temperature, and was washed 3 times with 1 mL PBS; and at last, each well was added with 0.5 mL of PBS and then observed and photographed with a Leica DM16000B inverted fluorescence microscope.

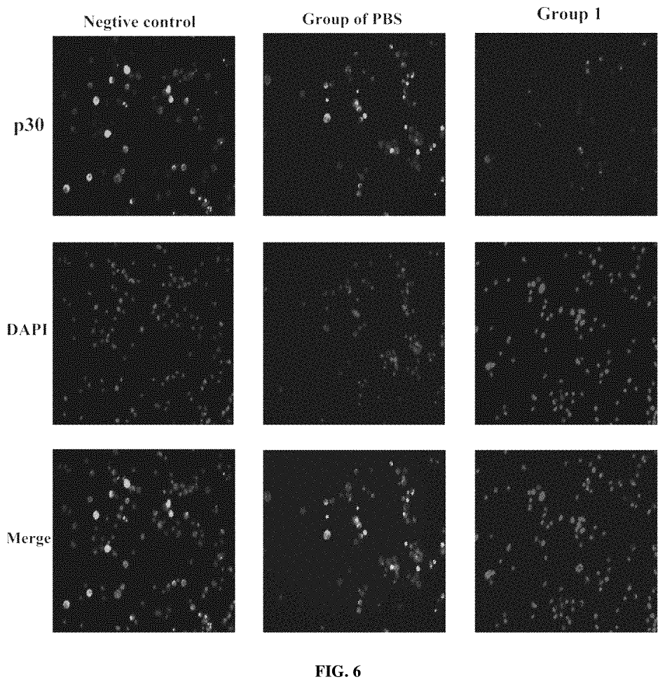

As shown in FIG. 6, compared with the negative control, the serum from pigs with vaccinated with the cocktail vaccine significantly reduced the TRITC fluorescence intensity and the number of positive cells after incubation with ASFV, while the TRITC fluorescence intensity and the number of positive cells in the PBS immunized group were not significantly changed. The results suggest that the antibodies induced by the cocktail vaccine can inhibit the infection of alveolar macrophage cells by ASFV.

2.5.5. Virus Neutralization Experiment

The serum from 0 and 42 d after the primary immunization was diluted at 1:5, filtered and sterilized with a 0.22 μm syringe filter, and then inactivated at 56° C. for 30 min; the serum was mixed with an equal volume of ASFV (MOI=0.01) and incubated overnight at 37° C. A serum/virus mixture was inoculated with a monolayer of porcine alveolar macrophages (PAM) (24-well plate), at 200 μL per well, incubated at 37° C., 5% CO₂ for 1 h. The mixture of serum and virus was discarded and washed 3 times with PBS; each well was added with 500 μL of RPMI-1640 containing 5% FBS, and incubated at 37° C., 5% CO₂ for 48 h; the cells were collected, and ASFV genomes of each experimental group were extracted by a DNA genome extraction kit; a qPCR kit was used to amplify the ASFV gene, the number of ASFV genome copies in the sample was calculated, and a virus neutralization rate was indirectly calculated: virus neutralization rate (%)=100-100*ASFV copy number after incubation with immune serum/ASFV copy number after incubation with pre-immune serum.

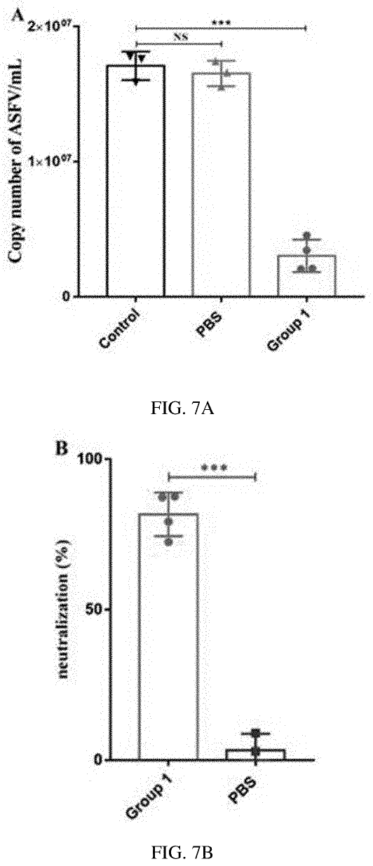

As shown in FIG. 7A, compared with the pre-immune serum (control group) and the PBS group, the serum from pigs vaccinated with the cocktail vaccine significantly reduced the copy number of ASFV in PAM cells (p<0.001), and had a significant inhibitory effect on the ASFV (p<0.001).

With the pre-immune serum as a control, the inhibition rate of the immune serum from the vaccine group against ASFV infection was 81.6% (FIG. 7B). The results indicated that the cocktail vaccine induces protective neutralizing antibodies in pigs after immunizing, which blocked ASFV infection of PAM cells in vitro, and is a very promising vaccine.

It should be noted that the above embodiments should be construed as illustrative rather than limiting the claimed scope of the present disclosure. The claimed scope of the present disclosure is subject to the claims. For those skilled in the art, without departing from the spirit and scope of the present disclosure, some non-essential improvements and adjustments made still belong to the claimed scope of the present disclosure.
Sequence Listing Information:
   DTD Version: V1_3
   File Name: HLP20220602625.xml
   Software Name: WIPO Sequence
   Software Version: 2.0.0
   Production Date: 2022 Aug. 19
General Information:
   Current application/Applicant file reference: HLP20220602625
   Earliest priority application/IP Office: CN
   Earliest priority application/Application number: 202210024618.6
   Earliest priority application/Filing date: 2022-01-11
   Applicant name: Lanzhou Veterinary Research Institute, Chinese Academy of Agricultural Sciences
   Applicant name/Language: en
   Invention title: COCKTAIL VACCINE OF RECOMBINANT AFRICAN SWINE FEVER VIRUS (ASFV) ANTIGEN AND USE THEREOF (en)
   Sequence Total Quantity: 7
Sequences:
   Sequence Number (ID): 1
   Length: 1176
   Molecule Type: DNA
   Features Location/Qualifiers:
      source, 1 . . . 1176
         >mol_type, other DNA
         >note, Nucleotide sequence encoding the PM
         >organism, synthetic construct
Residues:

atggacttca ttctgaacat cagcatgaag atggaagtta tcttcaagac cgacctgcgt 60
agcagcagcc aagtggtttt ccacgcgggt agcctgtaca actggtttag cgttgagatc 120
attaacagcg gccgtattgt gaccaccgcg atcaaaaccc tgctgagcac cgtgaagtat 180
gacattgtta aaagcgcgcg tatctacgcg ggtcagggct ataccgaaca ccaggcgcaa 240
gaggaatgga acatgattct gcacgtgctg ttcgaggaag agaccgagag cagcgcgagc 300
agcgaaaaca tccacgagaa gaacgataac gaaaccaacg agtgcaccag cagcttcgaa 360
accctgtttg aacaagagcc gagcagcgag gttccgaagg acagcaaact gtacatgctg 420
gcgcagaaaa ccgtgcaaca cattgaacag tatggcaagg cgccggattt caacaaagtt 480
atccgtgcgc acaactttat tcagaccatc tacggcaccc cgctgaagga agaggaaaaa 540
gaggtggttc gtctgatggt gatcaagctg ctgaagaaaa agggtggcgg tggcagcggt 600
ggcggtggca gcggtggcgg tggcagcatg gatagcgaat tcatggatag tgaatttttc 660
cagccggtgt atccgcgcca ttatggcgaa tgtctgagtc cggtgaccac cccgagcttt 720
tttagcaccg gtggcggtgg cagcggtggc ggtggcagcg gtggcggtgg cagcagcagc 780
cgcaaaaaaa aagcagccgc aattgaagaa gaagatattc agtttatcaa cccgtatcag 840
gatcagcagt gggttgaagt gaccccgcag ccgggtacca gtaaaccggc aggtgcaacc 900
accgcaagtg tgggtaaacc ggtgaccggc cgtccggcaa ccaatcgtcc ggccaccaat 960
aaaccggtta ccgataatcc ggttaccgac cgcctggtga tggccaccgg tggtccggca 1020
gcagcaccgg cagcagctag tgcaccggca catccggcag aaccgtatac caccgtgacc 1080
acccagaata ccgccagtca gaccatgagc gccattgaaa atctgcgtca gcgtaatacc 1140
tatacccata aagatctgga aaatagtctg ctcgag 1176

Sequence Number (ID): 2
Length: 1206
Molecule Type: DNA
Features Location/Qualifiers:
   source, 1 . . . 1206
      >mol_type, other DNA
      >note, Nucleotide sequence encoding the PPE
      >organism, synthetic construct
Residues:

atgcagaaag acctggttaa cgaattcccg ggtctgttcg ttcgtcagtc tcgtttcatc 60
gcgggtcgtc cgtctcgtcg taacatccgt ttcaaaccgg gtggtggtgg ttctggtggt 120
ggtggttctg gtggtggtgg ttctgcgtgc tcttctatct ctgacatctc tccggttacc 180
tacccgatca ccctgccgat catcaaaaac atctctgtta ccgcgcacgg tatcaacctg 240
atcgacaaag gtggtggtgg ttctggtggt ggtggttctg gtggtggtgg ttcttactgc 300
gaatacccgg gtgaacgtct gtacgaaaac gttcgtttcg acgttaacgg taactctctg 360
gacgaatact cttctgacgt taccaccctg ccgggtctga aaccgcgtga agaataccag 420
ccgtctggtg gtggtggttc tggtggtggt ggttctggtg gtggtggttc tctgtgcaac 480
atccacgacc tgcacaaacc gcaccagtct aaaccgatcc tgaccgacga aaacgacacc 540
cagcgtacct gctctcacac caacccgggt ggtggtggtt ctggtggtgg tggttctggt 600
ggtggtggtt ctatgggtgg ttctacctct aaaaactctt tcaaaaacac caccaacatc 660
atctctaact ctatcttcaa ccagatgcag tcttgcatct ctatgctgga cggtaaaaac 720
tacatcggtg ttttcggtga cggtaacatc ctgaaccacg ttttccagga cctgaacctg 780
tctctgaaca cctcttgcgt tcagaaacac gttaacgaag aaaacttcat caccaacctg 840
tctaaccaga tcacccagaa cctgaaagac caggaagttg cgctgaccca gtggatggac 900
gcgggtaccc acgaccagaa aaccgacatc gaagaaaaca tcaaagttaa cctgaccacc 960
accctgatcc agaactgcgt ttcttctctg tctggtatga acgttctggt tgttaaaggt 1020
aacggtaaca tcgttgaaaa cgcgacccag aaacagtctc agcagatcat ctctaactgc 1080
ctgcagggtt ctaaacaggc gatcgacacc accaccggta tcaccaacac cgttaaccag 1140
tactctcact acacctctaa aaacttcttc gacttcatcg cggacgcgat ctctgcggtt 1200
ttcaaa 1206

Sequence Number (ID): 3
Length: 945
Molecule Type: DNA
Features Location/Qualifiers:
   source, 1 . . . 945
      >mol_type, other DNA
      >note, The nucleotide sequence encoding the CPE
      >organism, synthetic construct
Residues:

atggactact gggtttcttt caacaaaacc atcatcctgg actctaacat caccaacgac 60
aacaacgaca tcaacggtgt ttcttggaac ttcttcaaca actctttcaa caccctggcg 120
acctgcggta aagcgggtaa cttctgcgaa tgctctaact actctacctc tatctacaac 180
atcaccaaca actgctctct gaccatcttc ccgcacaacg acgttttcga caccacctac 240
caggttgttt ggaaccagat catcaactac accatcaaac tgctgacccc ggcgaccccg 300
ccgaacatca cctacaactg caccaacttc ctgatcacct gcaaaaaaaa caacggcacc 360
aacaccaaca tctacctgaa catcaacgac accttcgtta aatacaccaa cgaatctatc 420
ctggaataca actggaacaa ctctaacatc aacaacttca ccgcgacctg catcatcaac 480
aacaccatct ctacctctaa cgaaaccacc ctgatcaact gcacctacct gaccctgtct 540
tctaactact tctacacctt cttcaaactg tacgagctcg gtggtggtgg ttctggtggt 600
ggtggttctc atatgaacaa accgatctgc taccagaacg acgacaaaat cttctactgc 660
ccgaaagact gggttggtta caacaacgtt tgctactact tcggtaacga agaaaaaaac 720
tacaacaacg cgtctaacta ctgcaaacag ctgaactcta ccctgaccaa caacaacacc 780
atcctggtta acctgaccaa aaccctgaac ctgaccaaaa cctacaacca cgaatctaac 840
tactgggtta actactctct gatcaaaaac gaatctgttc tgctgcgtga ctctggttac 900
tacaaaaaac agaaacacgt ttctctgctg tacatctgct ctaaa 945

Sequence Number (ID): 4
Length: 392
Molecule Type: AA
Features Location/Qualifiers:
   source, 1 . . . 392
      >mol_type, protein
      >note, Recombinant ASFV antigen fusion proteins PM
      >organism, synthetic construct
Residues:

MDFILNISMK MEVIFKTDLR SSSQVVFHAG SLYNWFSVEI INSGRIVTTA IKTLLSTVKY 60
DIVKSARIYA GQGYTEHQAQ EEWNMILHVL FEEETESSAS SENIHEKNDN ETNECTSSFE 120
TLFEQEPSSE VPKDSKLYML AQKTVQHIEQ YGKAPDFNKV IRAHNFIQTI YGTPLKEEEK 180
EVVRLMVIKL LKKKGGGGSG GGGSGGGGSM DSEFMDSEFF QPVYPRHYGE CLSPVTTPSF 240
FSTGGGGSGG GGSGGGGSSS RKKKAAAIEE EDIQFINPYQ DQQWVEVTPQ PGTSKPAGAT 300
TASVGKPVTG RPATNRPATN KPVTDNPVTD RLVMATGGPA AAPAAASAPA HPAEPYTTVT 360
TQNTASQTMS AIENLRQRNT YTHKDLENSL LE 392

Sequence Number (ID): 5
Length: 402
Molecule Type: AA
Features Location/Qualifiers:
   source, 1 . . . 402
      >mol_type, protein
      >note, Recombinant ASFV antigen fusion proteins PPE
      >organism, synthetic construct
Residues:

MQKDLVNEFP GLFVRQSRFI AGRPSRRNIR FKPGGGGSGG GGSGGGGSAC SSISDISPVT 60
YPITLPIIKN ISVTAHGINL IDKGGGGSGG GGSGGGGSYC EYPGERLYEN VRFDVNGNSL 120
DEYSSDVTTL PGLKPREEYQ PSGGGGSGGG GSGGGGSLCN IHDLHKPHQS KPILTDENDT 180
QRTCSHTNPG GGGSGGGGSG GGGSMGGSTS KNSFKNTTNI ISNSIFNQMQ SCISMLDGKN 240
YIGVFGDGNI LNHVFQDLNL SLNTSCVQKH VNEENFITNL SNQITQNLKD QEVALTQWMD 300
AGTHDQKTDI EENIKVNLTT TLIQNCVSSL SGMNVLVVKG NGNIVENATQ KQSQQIISNC 360
LQGSKQAIDT TTGITNTVNQ YSHYTSKNFF DFIADAISAV FK 402

Sequence Number (ID): 6
Length: 315
Molecule Type: AA
Features Location/Qualifiers:
   source, 1 . . . 315
      >mol_type, protein
      >note, Recombinant ASFV antigen fusion proteins CPE
      >organism, synthetic construct
Residues:

MDYWVSFNKT IILDSNITND NNDINGVSWN FFNNSFNTLA TCGKAGNFCE CSNYSTSIYN 60
ITNNCSLTIF PHNDVFDTTY QVVWNQIINY TIKLLTPATP PNITYNCTNF LITCKKNNGT 120
NTNIYLNIND TFVKYTNESI LEYNWNNSNI NNFTATCIIN NTISTSNETT LINCTYLTLS 180
SNYFYTFFKL YELGGGGSGG GGSHMNKPIC YQNDDKIFYC PKDWVGYNNV CYYFGNEEKN 240
YNNASNYCKQ LNSTLTNNNT ILVNLTKTLN LTKTYNHESN YWVNYSLIKN ESVLLRDSGY 300

Sequence Number (ID): 7
Length: 5
Molecule Type: AA
Features Location/Qualifiers:
   source, 1 . . . 5
      >mol_type, protein
      >note, The sequence of the linker
      >organism, synthetic construct
Residues:

GGGGS5 5

END

What is claimed is:

1. A cocktail vaccine of a recombinant African swine fever virus (ASFV) antigen, wherein the cocktail vaccine comprises two or more of three recombinant ASFV antigen fusion proteins PM, PPE, and CPE as active ingredients;
the PM is formed by fusion of an ASFV p30 and a modified p54 through a Linker, with a general formula of p30-(Linker)₃-mp54;
the PPE is formed by fusion of four verified epitopes of an ASFV p72 and an N-segment amino acid sequence of pE248R through the Linker, with a general formula of p72 epitope 1-(Linker)₃-p72 epitope 2-(Linker)₃-p72 epitope 3-(Linker)₃-p72 epitope 4-(Linker)₃-N-segment amino acid sequence of pE248R;
the CPE is formed by fusion of an N-segment amino acid sequence of CD2v and a C-segment amino acid sequence of pEP153R through the Linker, with a general formula of N-segment amino acid sequence of CD2v-(Linker)₂-C-segment amino acid sequence of pEP153R; and
the Linker has the sequence set forth in SEQ ID NO:7.

2. The cocktail vaccine according to claim 1, wherein the PM has the amino acid sequence set forth in SEQ ID NO: 4; the PPE has the amino acid sequence set forth in SEQ ID NO: 5; and the CPE has the amino acid sequence set forth in SEQ ID NO: 6.

3. The cocktail vaccine according to claim 2, wherein the nucleotide sequence encoding the PM is set forth in SEQ ID NO: 1; the nucleotide sequence encoding the PPE is set forth in SEQ ID NO: 2; and the nucleotide sequence encoding the CPE is set forth in SEQ ID NO: 3.

4. The cocktail vaccine according to claim 1, wherein the cocktail vaccine comprises three recombinant ASFV antigen fusion proteins PM, PPE, and CPE as active ingredients.

5. The cocktail vaccine according to claim 4, further comprising an adjuvant, wherein the cocktail vaccine is prepared by mixing the PM, the PPE, and the CPE with the adjuvant.

6. The cocktail vaccine according to claim 5, wherein the three recombinant ASFV antigen fusion proteins PM, PPE, and CPE are mixed in a mass ratio of 1:1:2 to obtain an antigen mixture; and the antigen mixture is emulsified into the cocktail vaccine with equal quality ISA 206 adjuvant.

7. A method for preparing a drug for treating and preventing ASFV infection, wherein comprises using the cocktail vaccine according to claim 1.

8. The cocktail vaccine according to claim 4, wherein the PM has the amino acid sequence set forth in SEQ ID NO: 4; the PPE has the amino acid sequence set forth in SEQ ID NO: 5; and the CPE has the amino acid sequence set forth in SEQ ID NO: 6.


SEQUENCE LISTING (7 sequences)

SEQ ID NO: 1 - DNA, 1176 bp, synthetic construct
  source 1..1176: mol_type = other DNA; note = Nucleotide sequence encoding the PM
atggacttcattctgaacatcagcatgaagatggaagttatcttcaagaccgacctgcgtagcagcagccaagtggttttccacgcgggtagcctgtacaactggtttagcgttgagatcattaacagcggccgtattgtgaccaccgcgatcaaaaccctgctgagcaccgtgaagtatgacattgttaaaagcgcgcgtatctacgcgggtcagggctataccgaacaccaggcgcaagaggaatggaacatgattctgcacgtgctgttcgaggaagagaccgagagcagcgcgagcagcgaaaacatccacgagaagaacgataacgaaaccaacgagtgcaccagcagcttcgaaaccctgtttgaacaagagccgagcagcgaggttccgaaggacagcaaactgtacatgctggcgcagaaaaccgtgcaacacattgaacagtatggcaaggcgccggatttcaacaaagttatccgtgcgcacaactttattcagaccatctacggcaccccgctgaaggaagaggaaaaagaggtggttcgtctgatggtgatcaagctgctgaagaaaaagggtggcggtggcagcggtggcggtggcagcggtggcggtggcagcatggatagcgaattcatggatagtgaatttttccagccggtgtatccgcgccattatggcgaatgtctgagtccggtgaccaccccgagcttttttagcaccggtggcggtggcagcggtggcggtggcagcggtggcggtggcagcagcagccgcaaaaaaaaagcagccgcaattgaagaagaagatattcagtttatcaacccgtatcaggatcagcagtgggttgaagtgaccccgcagccgggtaccagtaaaccggcaggtgcaaccaccgcaagtgtgggtaaaccggtgaccggccgtccggcaaccaatcgtccggccaccaataaaccggttaccgataatccggttaccgaccgcctggtgatggccaccggtggtccggcagcagcaccggcagcagctagtgcaccggcacatccggcagaaccgtataccaccgtgaccacccagaataccgccagtcagaccatgagcgccattgaaaatctgcgtcagcgtaatacctatacccataaagatctggaaaatagtctgctcgag

SEQ ID NO: 2 - DNA, 1206 bp, synthetic construct
  source 1..1206: mol_type = other DNA; note = Nucleotide sequence encoding the PPE
atgcagaaagacctggttaacgaattcccgggtctgttcgttcgtcagtctcgtttcatcgcgggtcgtccgtctcgtcgtaacatccgtttcaaaccgggtggtggtggttctggtggtggtggttctggtggtggtggttctgcgtgctcttctatctctgacatctctccggttacctacccgatcaccctgccgatcatcaaaaacatctctgttaccgcgcacggtatcaacctgatcgacaaaggtggtggtggttctggtggtggtggttctggtggtggtggttcttactgcgaatacccgggtgaacgtctgtacgaaaacgttcgtttcgacgttaacggtaactctctggacgaatactcttctgacgttaccaccctgccgggtctgaaaccgcgtgaagaataccagccgtctggtggtggtggttctggtggtggtggttctggtggtggtggttctctgtgcaacatccacgacctgcacaaaccgcaccagtctaaaccgatcctgaccgacgaaaacgacacccagcgtacctgctctcacaccaacccgggtggtggtggttctggtggtggtggttctggtggtggtggttctatgggtggttctacctctaaaaactctttcaaaaacaccaccaacatcatctctaactctatcttcaaccagatgcagtcttgcatctctatgctggacggtaaaaactacatcggtgttttcggtgacggtaacatcctgaaccacgttttccaggacctgaacctgtctctgaacacctcttgcgttcagaaacacgttaacgaagaaaacttcatcaccaacctgtctaaccagatcacccagaacctgaaagaccaggaagttgcgctgacccagtggatggacgcgggtacccacgaccagaaaaccgacatcgaagaaaacatcaaagttaacctgaccaccaccctgatccagaactgcgtttcttctctgtctggtatgaacgttctggttgttaaaggtaacggtaacatcgttgaaaacgcgacccagaaacagtctcagcagatcatctctaactgcctgcagggttctaaacaggcgatcgacaccaccaccggtatcaccaacaccgttaaccagtactctcactacacctctaaaaacttcttcgacttcatcgcggacgcgatctctgcggttttcaaa

SEQ ID NO: 3 - DNA, 945 bp, synthetic construct
  source 1..945: mol_type = other DNA; note = The nucleotide sequence encoding the CPE
atggactactgggtttctttcaacaaaaccatcatcctggactctaacatcaccaacgacaacaacgacatcaacggtgtttcttggaacttcttcaacaactctttcaacaccctggcgacctgcggtaaagcgggtaacttctgcgaatgctctaactactctacctctatctacaacatcaccaacaactgctctctgaccatcttcccgcacaacgacgttttcgacaccacctaccaggttgtttggaaccagatcatcaactacaccatcaaactgctgaccccggcgaccccgccgaacatcacctacaactgcaccaacttcctgatcacctgcaaaaaaaacaacggcaccaacaccaacatctacctgaacatcaacgacaccttcgttaaatacaccaacgaatctatcctggaatacaactggaacaactctaacatcaacaacttcaccgcgacctgcatcatcaacaacaccatctctacctctaacgaaaccaccctgatcaactgcacctacctgaccctgtcttctaactacttctacaccttcttcaaactgtacgagctcggtggtggtggttctggtggtggtggttctcatatgaacaaaccgatctgctaccagaacgacgacaaaatcttctactgcccgaaagactgggttggttacaacaacgtttgctactacttcggtaacgaagaaaaaaactacaacaacgcgtctaactactgcaaacagctgaactctaccctgaccaacaacaacaccatcctggttaacctgaccaaaaccctgaacctgaccaaaacctacaaccacgaatctaactactgggttaactactctctgatcaaaaacgaatctgttctgctgcgtgactctggttactacaaaaaacagaaacacgtttctctgctgtacatctgctctaaa

SEQ ID NO: 4 - protein, 392 aa, synthetic construct
  source 1..392: note = Recombinant ASFV antigen fusion proteins PM
MDFILNISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIKLLKKKGGGGSGGGGSGGGGSMDSEFMDSEFFQPVYPRHYGECLSPVTTPSFFSTGGGGSGGGGSGGGGSSSRKKKAAAIEEEDIQFINPYQDQQWVEVTPQPGTSKPAGATTASVGKPVTGRPATNRPATNKPVTDNPVTDRLVMATGGPAAAPAAASAPAHPAEPYTTVTTQNTASQTMSAIENLRQRNTYTHKDLENSLLE

SEQ ID NO: 5 - protein, 402 aa, synthetic construct
  source 1..402: note = Recombinant ASFV antigen fusion proteins PPE
MQKDLVNEFPGLFVRQSRFIAGRPSRRNIRFKPGGGGSGGGGSGGGGSACSSISDISPVTYPITLPIIKNISVTAHGINLIDKGGGGSGGGGSGGGGSYCEYPGERLYENVRFDVNGNSLDEYSSDVTTLPGLKPREEYQPSGGGGSGGGGSGGGGSLCNIHDLHKPHQSKPILTDENDTQRTCSHTNPGGGGSGGGGSGGGGSMGGSTSKNSFKNTTNIISNSIFNQMQSCISMLDGKNYIGVFGDGNILNHVFQDLNLSLNTSCVQKHVNEENFITNLSNQITQNLKDQEVALTQWMDAGTHDQKTDIEENIKVNLTTTLIQNCVSSLSGMNVLVVKGNGNIVENATQKQSQQIISNCLQGSKQAIDTTTGITNTVNQYSHYTSKNFFDFIADAISAVFK

SEQ ID NO: 6 - protein, 315 aa, synthetic construct
  source 1..315: note = Recombinant ASFV antigen fusion proteins CPE
MDYWVSFNKTIILDSNITNDNNDINGVSWNFFNNSFNTLATCGKAGNFCECSNYSTSIYNITNNCSLTIFPHNDVFDTTYQVVWNQIINYTIKLLTPATPPNITYNCTNFLITCKKNNGTNTNIYLNINDTFVKYTNESILEYNWNNSNINNFTATCIINNTISTSNETTLINCTYLTLSSNYFYTFFKLYELGGGGSGGGGSHMNKPICYQNDDKIFYCPKDWVGYNNVCYYFGNEEKNYNNASNYCKQLNSTLTNNNTILVNLTKTLNLTKTYNHESNYWVNYSLIKNESVLLRDSGYYKKQKHVSLLYICSK

SEQ ID NO: 7 - protein, 5 aa, synthetic construct
  source 1..5: note = The sequence of the linker
GGGGS